(19) United States
(12) Patent Application Publication
(10) Pub. No.: US 2017/0021122 A1
(43) Pub. Date: Jan. 26, 2017

(54) FILTER HEAT AND MOISTURE EXCHANGE DEVICE

(71) Applicant: Pawel Wisniewski, Durban (ZA)

(72) Inventor: Pawel Wisniewski, Durban (ZA)

(21) Appl. No.: 15/039,834

(22) PCT Filed: Dec. 5, 2014
(86) PCT No.: PCT/ZA2014/000069

§ 371 Date: May 26, 2016

(30) Foreign Application Priority Data
Dec. 5, 2013 (ZA) 2013/08652

(51) Int. Cl. A61M 16/08; A61B 5/097; A61M 16/06; A61M 16/00; A61M 16/04; A61M 16/10; A61M 16/01

(52) U.S. Cl. CPC A61M 16/085; A61M 16/1055; A61M 16/1045; A61M 16/0875; A61B 5/097; A61M 16/01; A61M 16/0045; A61M 16/04; A61M 16/06; A61M 2230/432

(57) ABSTRACT

Filtered heat and moisture exchange device for a breathing system comprises a housing (40) having first and second ports (42,46) connectable to breathing lines (20,22) and a chamber (50) being located between and in flow communication with the ports. An air filter (52) and a heat and moisture exchange means (54) are located in the chamber (50). A tubular gas sampling member (64) is extending from gas sampling port (56) located near the first port (42) to a sampling location (B) in the second port (46) by passing chamber (50) and central apertures of air filter (52) and heat and moisture exchange means (54).

### FIELD OF THE INVENTION

This invention relates to a filter, heat and moisture exchange device for use in a patient breathing circuit, particularly for capnography.

### BACKGROUND OF INVENTION

Breaking systems and breathing circuits are well known to provide respiratory gas or air to patients who experience difficulty breathing autonomously, for example, when a patient is under an anaesthetic, for example, during a medical procedure. Though a breathing system supplies air to the lungs of a patient, the gas or air supplied is typically not at a suitable temperature and/or humidity for effective use in the patient's lungs and may in certain instances contain impurities or microorganisms. In this regard filter heat and moisture exchanger (FHME) devices are inserted in flow connection the breathing systems between the breathing systems and the patients, to address the aforementioned drawbacks. In particular, the FHME devices filter air supplied to/inhaled by the patient and also retains at least some of the heat and moisture from the air exhaled by the patient and transfers the same to the air inhaled by the patient.

A conventional FHME device typically comprises a housing having a first port located at first end portion of the housing distal to the patient, a second port located at a second end portion of the housing proximal to the patient, and a chamber housing a filter and suitable heat and moisture exchange means located between and in flow communication with the first and second ports. The first port is usually connected via tubing to a respirator apparatus in the breathing circuit and the second port is usually connected to the patient, for example, directly to the patient's airways by means of an endotracheal tube, laryngeal mask, facial mask, or the like. As FHME devices are located usually as close as possible to the patient's airways, they are often provided with gas sampling ports via which at least carbon dioxide levels of a patient can be measured by conventional means for capnography.

One drawback with some known conventional FHME devices is that the gas sampling ports are located at regions of the housing which provide poor carbon dioxide measurements/capnographic data when high flows of anaesthetic gases are used as it often is pediatric patients. For example, a gas sampling port located adjacent first end portion facilitates sampling (via a suitable gas sampling apparatus connected to the gas sampling port) of exhaled air from a patient downstream from the filer and heat and moisture exchange means which results in poor or inaccurate measurements being obtained as the filter and/or the means interferes with the carbon dioxide content of the air sampled. Incorrect or poor carbon dioxide measurements are undesirable in instances where capnography is important, for example, when the patient is under an anaesthetic during a medical procedure and when higher anaesthetic gas flows are used as it is in pediatric patients. It will be appreciated that in adult patients gas flows are lower resulting in sharper capnograph traces and a greater problem exists for pediatric patients where higher anaesthetic gas flows are used.

To address the above described drawback some conventional FHME devices provide gas sampling ports, upstream from the filter, heat and moisture exchange means in receiving exhaled breath from a patient. For example, gas sampling ports located adjacent the second end portion of the device as described above yield more accurate carbon dioxide measurements as exhaled breath is sampled proximal to the patient upstream from the filter, and heat and moisture exchange means. However, the drawback of these devices is that, in use, the gas sampling ports located adjacent the second end portions are difficult and/or impractical to reach to connect appropriate sensing apparatuses thereto as the second end portions are usually in very close proximity to mouths of patients, especially when tubing from the second ports are located in the throats of the patients. It follows that in these applications a gas sampling port located adjacent the second end portion proximal to the mouth of a patient may be obstructive, uncomfortable, impractical, and/or inconvenient especially when connecting the external gas sampling apparatus thereto. Such ports also interfere with and make connection of airway securing devices difficult.

It will be appreciated that increasing the length of tubing from the device to the patient may address the last mentioned drawback as sampling port can be accessed easier. However, this is inherently undesirable as it is important to keep the device in relatively close proximity to the patient to decrease the overall dead zone in the breathing circuit to the patient.

Moreover, the drawbacks relating to the placement of the gas sampling port proximal to the patient is exacerbated in pediatric applications where the device is to be used with an infant/chid. In pediatric applications, it is desirous to have the devices as close as possible to the mouth of the patient so as to avoid unnecessarily introducing the length of the dead zone in the breathing circuit to the patient. It follows that a gas sampling port adjacent a second end portion of the device for pediatric applications is therefore not ideal.

It is amongst others, an object of the present invention at least to address some of the above mentioned drawbacks and provide at least an alternative filter, heat and moisture exchange device.

### BRIEF DESCRIPTION OF THE DRAWINGS

FIG. 1 shows a schematic diagram of at least a substantial portion of a ventilation or breathing circuit including a filter, heat and moisture exchange (FHME) device, in accordance with an example embodiment of the invention, as well as carbon dioxide trace curve graphs;

FIG. 2 shows a three dimensional view of a FHME device in accordance with an example embodiment of the invention;

FIG. 3 shows a three dimensional exploded view of a FHME device in accordance with an example embodiment of the invention;

FIG. 4 shows a sectional view through a FHME device in accordance with a specific example embodiment of the invention; and

FIG. 5 shows another sectional view through a FHME device in accordance with another specific example embodiment of the invention.

### DESCRIPTION OF PREFERRED EMBODIMENTS

In the following description, for purposes of explanation, numerous specific details are set forth in order to provide a thorough understanding of an embodiment of the present disclosure. It will be evident, however, to one skilled in the art that the present disclosure may be practiced without these specific details.

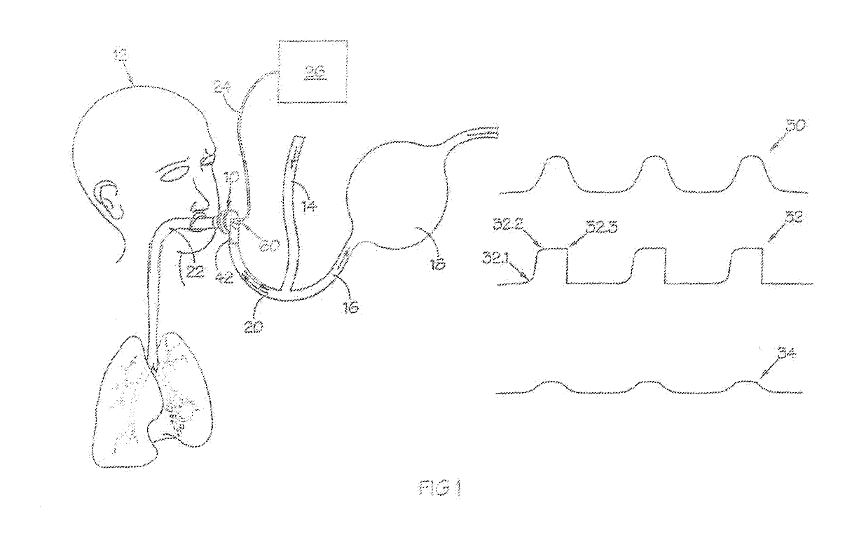

Referring to FIGS. 1 to 5 of the drawings a filter heat and moisture exchange (FHME) device in accordance with an example embodiment of the invention is generally located by reference numeral 10.

Although reference will be made to the FIGS. 1 to 5 in general, it will be appreciated that attention may be drawn to one or more of the drawings, as the case may be, in particular to facilitate better understanding of the invention described herein.

The device 10 is typically for use in a conventional breathing system or breathing circuit for ventilating a patient 12 with a gas (e.g. air or oxygenated air) when the patient 12 is incapacitated and cannot breathe unaided such as when the patient 12 is in a coma or is anaesthetized such as during a medical procedure, or the like. It follows that the system may also supply the patient 12 with anaesthetic gas for inhalation thereby to anaesthetize the same in a conventional manner. A portion of an example breathing circuit is illustrated in FIG. 1. As can be seen, the device 10 is locatable within the breathing circuit proximal to a patient 12 being ventilated. The device 10 serves at least to filter air supplied to/inhaled by the patient and also retain at least some of the heat and moisture from the air exhaled by the patient and transfer the same to the air inhaled by the patient 12, in use, in a conventional manner thereby to assist the patient 12 in breathing.

Though not illustrated and described in detail, it will be noted that the illustrated portion of the breathing circuit comprises a gas supply line 14 supplying fresh or oxygenated air from a suitable ventilation apparatus, for inhalation by the patient, at a constant flow rate and a return line 16 transporting air exhaled by the patient 12 to a re-breathing bag 18. The breathing circuit further comprises a circuit tubing portion 20 connectable to the device 10 and in flow communication with fresh gas lines 14 and bag line 16; and an airway securing device 22 which is connectable between the device 10 and the patient 12, the device 22 not necessarily forming part of the breathing circuit.

It will be understood that the device 10 described herein may work with various conventional breathing systems, not illustrated. However, the portion of the breathing system illustrated serves to exemplify an example use of the device 10 in accordance with the invention.

The device 22 may fit into the mouth of the patient 12 and be receivable the airways of the patient 12 to ventilate the lungs of the same. In the case of infants and/or children it is desirable to have the device 10 fairly close to the mouth of the patient 12 or to the airway securing device 22 to avoid introducing a further dead zone in the breathing circuit thereto.

The airway securing device 22 may be an endotracheal tube or laryngeal mask. In some non-invasive example, embodiments (not shown) the device 10 may be connected to the patient via a face mask which fits over the mouth and nose of the patient 12.

A gas sampling line 24 connected to a gas sampling apparatus 26 is typically connectable to the device 10 to enable the apparatus 26 to measure, inter alia, carbon dioxide (CO₂) levels in the gas or air exhaled by the patient 12. In other words the exhaled gas is sampled for capnography for determining a condition of the patient 12 being ventilated. An important point to bear in mind in capnography is that CO₂ measurements vary depending on where sampling of exhaled gases takes place especially if the flow of gas through the tubing 14 is high as often is the case in pediatric anesthesiology. In this regard, reference is made to the illustrative CO₂ trace graphs/capnograms 30, 32, and 34 which correspond to CO₂ measured at various locations in the breathing circuit. For example, in graph 30, a CO₂ trace curve is illustrated for gas sampled at tube 20; in graph 32, a CO₂ trace curve is illustrated for gas sampled at device 22; and in graph 34, a carbon dioxide trace curve is illustrated for gas sampled at tube 18.

From the graphs 30, 32 and 34, it may be seen that graph 32 is most desirable as one may be able to extract more data regarding the respiration of the patient wherein point 32.1 represents the first portion of gas from the lungs of the patient 12 during exhalation as it contains alveolar gas with CO₂, 32.2 denotes a plateau, and point 32.3 indicates the begin of inhalation of fresh gas with measured CO₂ levels decreasing. Though slightly better than graph 34, graph 30 doesn't provide as much data as graph 32. In this regard follows that the most ideal location in the breathing system to sample gases for capnography is as close as possible to the lungs of the patient 12.

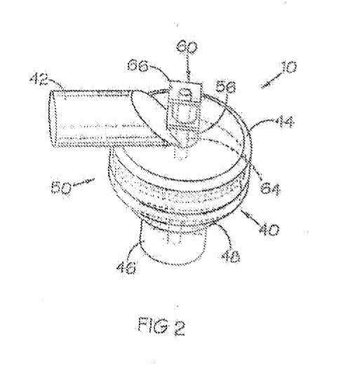

The device 10 is configured to this end and in this regard reference will now be made to FIGS. 2 to 5 in particular which illustrates the device 10 in accordance with an example embodiment in more detail.

The device 10 has a housing 40 having a first port 42 located a first end potion 44 of the housing 40 distal to the patient 12 in use; a second port 46 located at a second end portion 48 of the housing 40 proximal to the patent 12, in use; and a chamber 50 located between and in flow communication with the first and second ports 42, 46. The first and second ports 42, 46 may be substantially cylindrical protruding ports to which conventional flexible tubing used in breathing systems may be attachable, for example, with a taper on taper fit. In particular, the first port 42 is a breathing system facing port connectable to the breathing system via line 20 which is sealingly receivable within or around the first port 42 in a spigot socket fashion. The second port 46 is connectable to the respiratory system of the patient 12 via a device 22 (as shown in FIG. 1) in a similar fashion. As mentioned previously, the airways securing device 22 may be receivable in the airway of the patient 12.

Instead, the second port 46 may be connectable to the patient 12 via a suitable mask. It follows that the first port 42 is located distally from the patient 12 and the second port 46 is located proximally to the patient 12, in use.

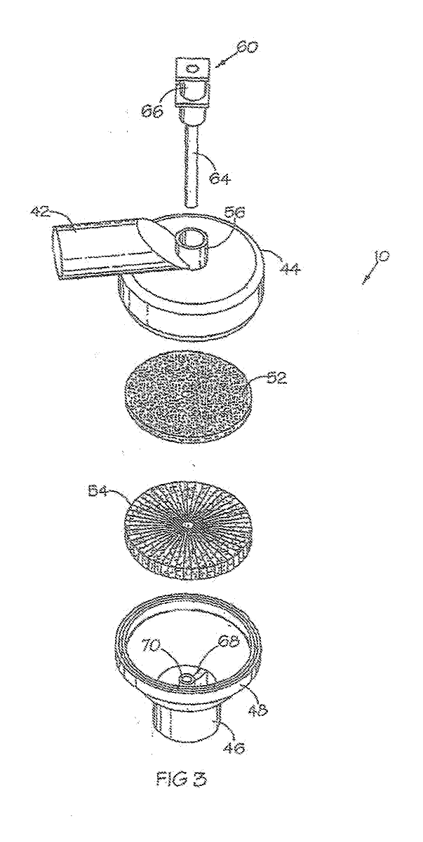

The chamber 50 is conveniently formed by the first end portion 44 and the second end portion 48 which may be in the form of plastic shells which are sealing and matingly attached at peripheries to define the chamber 50, as can be seen in FIG. 3 in this regard, the first end portion 44 may be referred to as the first chamber portion and the second end portion 48 as the second chamber portion. The first chamber portion 44 may be in the form of a substantially cylindrical shell having a circular platform and a skirt extending transversely from the periphery of the platform to engage with the second chamber portion 48. The second chamber portion 48 may be in the form of a substantially pad-spherical shell. In any event, the chamber 50 extends symmetrically about a central axis A. The second port 46 may be centered on the central axis A whereas the first port 42 may extend at least substantially transverse to the axis A.

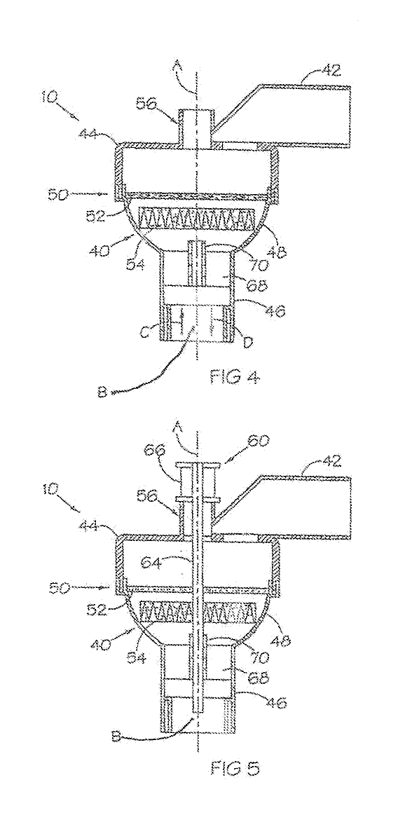

The first chamber portion 44 may define a peripherally extending lip extending from the skirt thereof which is matingly receivable within a circumferentially extending groove provided at a periphery of the second chamber portion 48 as can be seen in FIGS. 4 and 5. The lip may be attached to the groove by an adhesive, ultrasonic welding or the like.

A substantially circular filter 52 is fitted to the chamber 50 and is located above and abuts a heat and moisture exchange means 54 also located in the chamber 50. The means 54 is located in a seat or sump in the second chamber portion 48. The filter 52 may be a conventional air filter which filters impurities and bacteria, etc. from being inhaled by the patient 24. The means 54 may be shaped to fit substantially snugly in the second chamber portion 48 and may be a conventional heat and moisture exchange means 54, for example, comprising a plurality of webs and/or air-flow channels to retain at least some of the heat and moisture from air exhaled by the patient 12 and transfer the same to air inhaled by the patient 12.

The housing 40 also comprises a gas sampling port 56 centered on the axis A. The gas sampling port 56 may be a cylindrical protruding port axially spaced from the second port 46 along the axis A.

The device 10 also comprises a gas sampling means 60 having a sampling member 64 extending from the gas sampling port 56, in use, to a sampling location B in the second port 46. The sampling member 64 defines a sampling flow path between the gas sampling port 56 and the second port 46 of the housing 40, in use. To this end, the sampling member 64 is typically the form of a hollow needle or tube extending from the sampling port 56 to the second port 46 axially within the housing 40 such that an end thereof is located at the sampling location B. Sampling location B is proximate to the patient 12. Gas sampled from this location B provides desirable CO2 measurements such as those in the capnogram 32 as hereinbefore described. It will be noted that in a preferred example embodiment, the sampling location B is located adjacent an exit periphery of the second port 46 so as not to protrude or lie flush with the periphery of the second port 46. In this way, the end or tip of the sampling member does not pose a hazard for causing injury to the patient 12 or damaging equipment.

An opposite end of the sampling member 64 may define a luer lock 66 attachable to the gas sampling port 56 with a sealed fit. The luer lock 66 makes for easier attachment of the line 24 to the apparatus 26 and may be attached to the port 56 via adhesive means.

While not shown the sampling member 64 may be integral with the sampling port 56 with the luer lock 66 attached to the port 56 and the sampling member 64 forming part of the housing 40.

As more clearly shown in FIGS. 3, 4 and 5 the sampling member may removably fit the housing 40 that may comprise guide or locating means in the form of radially extending webs 68 and a collar 70 to locate the sampling member 64 securely, axially within the housing 40. It follows that the filter 52 and means 54 may define central apertures for receiving the member 64 there through such that the filter and the means 54 are seen to extend radially from the member 64. The filter 52, and optionally the means 54 may be sealingly engageable with the member 64 at the central aperture/s such that a substantially airtight seal is made there between. In this way, although the member 64 pierces through the filter 52, the integrity of the filtration is preserved as the filter 52 is sealed around the member 64.

In assembly, the means 54 is located in the second chamber portion 48, the filter 52 is located on the means 54 and the first chamber portion 44 is secured to the second chamber portion 48 substantially in a fashion as mentioned above. The sampling member 64 is introduced axially through the gas sampling port 56 and is guided and located by the locating means to the sampling location B. The member 64 is then secured to the port 56 via suitable means.

In use, referring to the drawings, the FHME device 10 is connected to a breathing circuit that is fitted to the patient 12 undergoing anaesthetic for a medical procedure. This is done by connecting tubing 20 to the first port 42, connecting the airways securing device 22 to the second port 46 and locating the device 22 in the throat (via the mouth) of the patient 12 in a conventional manner.

A sampling line 24 connected to conventional sampling apparatus 26 is connected to the luer lock 66 easily without causing disruption to the patient 12 and the breathing system especially when the device 10 is relatively close to the mouth of the patient 12 as is the case in certain pediatric applications.

The patient 12 breathes with gas inhaled flowing in direction D (FIG. 4) through the device 10 and gas exhaled by the patient flowing in direction C through the device 10. The filter 52 and means 54 serve to function in a conventional manner as the patient 12 breathes with the filter 52 function not being compromised due to the airtight sealing fit between the filter 52 and the member 64, at the center or central aperture of the filter 52.

While the patient 12 is under anaesthetic, capnography is performed with desirable CO₂ measurements being obtained from the location B which is proximal to the patient 12, particularly the lungs thereof, to enable medical practitioners to make more informed medical decisions during the medical procedure.

Being able to take a measurement from location B proximal to the patient 12 by accessing sampling port 54 (with the sampling line 24) at a location distal to the patient 12 is convenient and non-intrusive. In this way, the invention as hereinbefore described has the benefit of being able to obtain measurements from a location proximal to the patient 12 without the drawbacks and inconvenience of having to physically connect the sampling line to said location proximal to the patient 12 when the device 10 is close to the mouth of the patient 12.

The invention as hereinbefore described enables more accurate capnography to be performed on a patient undergoing artificial respiration. The invention also provides a more convenient and less obstructive means to obtain more accurate CO₂ measurements from a patient, proximal thereto. The invention finds particular application for use in pediatric applications whereby it is desirous to locate the device as described herein as close as possible to a mouth of an infant or child (to decrease the dead zone of the breathing circuit thereto) without causing discomfort to the infant or child in connecting the gas sampling apparatus thereto.

1-16. (canceled)

17. A filter heat and moisture exchange device for a breathing system, the device comprising:
a housing having a first port located at a first end portion of the housing distal to a patient, in use, a second port located at a second end portion of the housing proximal to the patient,
in use, a chamber located between and inflow communication with the first and second ports, and a gas sampling port located at the first end portion of the housing;
at least one of a filter and heat and moisture exchange means that is operatively situated in the chamber at least once the device is ready for use; and a gas sampling means,
characterized in that the gas sampling means has a sampling member extending from the gas sampling port to a sampling location (B) in the second port, at least once the device is ready for use, under which condition the sampling member defines a sampling flow path between the gas sampling port and the second port with the location of the gas sampling port facilitating the connection of gas sampling tubing thereto while retaining the location of sampling (B) proximal to a patient once the device is in operative use.

18. A filter, heat and moisture exchange device as claimed in claim 17 in which the sampling member is integral with the gas sampling port and, thus, the housing.

19. A filter, heat and moisture exchange device as claimed in claim 17 in which the first port is connectable to a breathing system and the second port to a respiratory system of the patient.

20. A filter, heat and moisture exchange device as claimed in claim 17 in which the sampling member is in the form of a hollow needle or a tube extending from the sampling port to the second port wherein an end of the tube is located at the sampling location.

21. A filter, heat and moisture exchange device as claimed in claim 17 in which the sampling member does not extend beyond a free end or a periphery of the second port.

22. A filter, heat and moisture exchange device as claimed in claim 17 in which the chamber comprises a first and a second chamber portions which are matingly engageable to define the substantially sealed chamber, the first and the second chamber portions defining the first end portion and the second end portion of the housing, respectively.

23. A filter, heat and moisture exchange device as claimed in claim 22 in which the heat and moisture exchange means is located in the second chamber portion with the filter abuttingly resting on the heat and moisture exchange means such that it is closer to the first chamber portion.

24. A filter, heat and moisture exchange device as claimed in claim 22 in which the chamber has a substantially bulbous shape while defining a central axis.

25. A filter, heat and moisture exchange device as claimed in claim 24 in which the gas sampling port, the second port and the sampling member are centered on the central axis.

26. A filter, heat and moisture exchange device as claimed in claim 25 in which the filter and the heat and moisture exchange means extend radially outwardly from the central axis.

27. A filter, heat and moisture exchange device as claimed in claim 26 in which the filter and the heat and moisture exchange means define central apertures to receive the sampling member axially therein.

28. A filter, heat and moisture exchange device as claimed in claim 27 in which the filter sealingly engages the sampling member having the effect of the sampling member piercing through both the filter and the heat and moisture exchange means such that at least the filter is located around the sampling member with a substantially snug or sealed fit.

29. A filter, heat and moisture exchange device as claimed in claim 24 in which the chamber defines locating means for locating the sampling member along the central axis.

30. A filter, heat and moisture exchange device as claimed in claim 17 in which the first port extends from the first chamber portion substantially transversely to the central axis.

31. A filter, heat and moisture exchange device as claimed in claim 17 in which the gas sampling port is sealingly connected to the sampling member at least once the device is ready for use.

32. A filter, heat and moisture exchange device as claimed in claim 31 in which the sampling means at least comprises a luer lock.
